(19) United States
(12) Patent Application Publication
(10) Pub. No.: US 2015/0231300 A1
(43) Pub. Date: Aug. 20, 2015

(54) PHARMACEUTICAL LIQUID ADHESIVE COMPOSITIONS FOR TREATMENT OF ANORECTAL DISORDERS

(71) Applicant: PERITECH PHARMA LTD., Herzliya (IL)

(72) Inventors: Evgenia Lozinsky, Beer Sheva (IL); Eran Eilat, Herzliya (IL)

(21) Appl. No.: 14/429,551

(22) PCT Filed: Oct. 27, 2013
(86) PCT No.: PCT/IL2013/050870

§ 371 Date: Mar. 19, 2015

Related U.S. Application Data
(60) Provisional application No. 61/719,419, filed on Oct. 28, 2012.

(51) Int. Cl. A61L 26/00

(52) U.S. Cl. CPC A61L 26/0019; A61L 26/0066; A61L 2300/45; A61L 2300/402

(57) ABSTRACT

The present invention provides liquid adhesive compositions comprising a silicone film forming agent, a volatile solvent and a pharmaceutically active agent, and methods for preparing said compositions. The invention further provides methods for treating anorectal disorders comprising administering the liquid adhesive compositions to the mucous anal surface, kits comprising the liquid adhesive composition and a container-applicator device.

### FIELD OF THE INVENTION

The present invention relates to pharmaceutical liquid adhesive compositions and uses thereof for treating anorectal disorders. Particularly, the present invention relates to liquid adhesive compositions comprising a silicone film forming agent, a volatile solvent, and a pharmaceutically active agent for use in treating hemorrhoids and other anorectal disorders.

### BACKGROUND OF THE INVENTION

Anorectal disorders are widespread and include a number of different conditions, such as hemorrhoids, fissures, abscesses, fistulae, and warts.

Various topically applied compositions for the treatment of anorectal conditions have been described, for example in U.S. Pat. Nos. 4,613,498; 4,626,433; 4,797,392; 5,166,132; 5,219,880; and 5,234,914.

Polymerizable tissue adhesives, also referred to as “liquid bandages” are synthetic adhesives comprising reactive monomer liquids that polymerize into a film when initiated by moisture or certain chemicals upon application to skin. The first liquid bandages proposed were based on monomers of cyanoacrylates, such as butyl-2-cyanoacrylate, which were surpassed by 2-octyl cyanocrylate which were found to yield superior product benefits such as rate of healing, and reduction of pain and infection.

Additional polymers used for liquid bandage technology have been based on other monomers, such as polyvinylpyrrolidones, pyroxylin/nitrocellulose, poly(methylacrylate-isobutene-monoisopropylmaleate), acrylates and siloxanes. In addition, combinations of different types of monomers have been included in liquid bandage polymer formulations.

Liquid bandages have been predominantly used for wound care as a replacement for conventional bandages. However, such bandages have also been used for closure of surgical wounds and in implantable devices.

U.S. Patent Application Publication No. 2002/0192273 discloses an adhesive patch for treating or preventing hemorrhoids which incorporates a therapeutic formulation comprising a vasoconstrictor, and optionally further comprising a polymer inter alia a polyacrylate, an analgesic, an anesthetic, an antipruritic, or a combination thereof.

U.S. Patent Application Publication No. 2006/0105028 discloses a system for treating warts, comprising a treatment formulation comprising a wart treating substance; and a cavity patch configured to become a closed cavity by application and adherence to a skin surface. According to U.S. 2006/0105028, the substance may be imiquimod, and the formulation may further include either or both of a gel-forming agent and a viscosity modifying agent, the latter of which may be a methacrylate polymer.

U.S. Pat. Nos. 4,987,893 and 5,103,812 disclose a liquid polymer-containing bandage material comprising siloxane containing polymer, and optionally further comprising an additional polymerizable comonomer selected from various acrylates; volatile polydimethylsiloxane liquid, and polar liquid; said bandage material is shown to be film forming at room temperature so as to form an adherent conformable moisture vapor permeable bandage. According to U.S. Pat. Nos. 4,987,893 and 5,103,812, the bandage material may be used for treating mucous membranes, inter alia hemorrhoids, and may incorporate medicaments.

U.S. Pat. No. 6,383,502 discloses non-stinging coating compositions comprising siloxane containing polymer, alkane-based siloxypolymer reaction solvent, and adjuvants, which compositions are useful for application to the skin or as components in cosmetic or topical medicament compositions.

U.S. Pat. No. 6,627,216 discloses a fluid composition for forming a patch in situ, the composition comprising a tacky component, such as a (meth)acrylate copolymer, and a film-forming non-tacky component which is preferably a siloxane containing polymer, such as a silicone polyurea or silicone polyurethane block polymer, wherein the composition may further contain a pharmacologically active agent.

U.S. Pat. No. 6,821,523 discloses a method of treating an individual afflicted with warts, comprising topically applying to an affected area of skin a formulation comprising a pharmaceutically acceptable topical carrier and a pharmacologically active base selected from inorganic hydroxides and nitrogenous bases, wherein the formulation may be in the form of a bioadhesive, such as a hydrogel and may further include imiquimod.

U.S. Pat. No. 7,318,937 discloses liquid coating compositions comprising siloxane containing polymer, volatile polydimethylsiloxane and aliphatic hydrocarbon. The compositions can be used for treating mucous membranes, inter alia hemorrhoids, and may incorporate medicaments.

There remains an unmet need for pharmaceutical liquid adhesive compositions which are effective, safe, and amenable to self-administration by a user.

### SUMMARY OF THE INVENTION

The present invention provides liquid adhesive compositions comprising active pharmaceutical agents and uses thereof for treating anorectal disorders, including hemorrhoids, anal fissures, anal warts, anal pruritis and other local anorectal lesions.

The liquid adhesive compositions of the present invention form a film on mucosal surfaces and thus provide a protective coating on irritated hemorrhoids and open fissures, resulting in protection of the laceration.

The present invention provides, in one aspect, a pharmaceutical liquid adhesive composition comprising a silicone film forming agent; a volatile solvent selected from the group consisting of a volatile polydimethylsiloxane, a volatile aliphatic hydrocarbon and a mixture thereof; and optionally at least one pharmaceutical agent.

In some embodiments, said silicone film forming agent is trimethylsiloxysilicate.

In some embodiments, said volatile polydimethylsiloxane is selected from the group consisting of hexamethyldisiloxane, octamethyl cyclotetrasiloxane, decamethyl cyclopentasiloxane, octamethyl trisiloxane, and mixtures thereof. Each possibility represents a separate embodiment of the present invention.

In some embodiments, said volatile aliphatic hydrocarbon is selected from the group consisting of alkanes, alkenes, alkynes, and mixtures thereof. Each possibility represents a separate embodiment of the present invention.

In some embodiments, said alkane is selected from the group consisting of pentane, isooctane, and mixtures thereof. Each possibility represents a separate embodiment of the present invention.

In some embodiments, said pharmaceutical agent is selected from the group consisting of an anesthetic agent, a vasoconstrictor, and combinations thereof. Each possibility represents a separate embodiment of the present invention.

In some embodiments, said anesthetic agent is selected from the group consisting of pramoxine, procaine, lidocaine, tetracaine, dibucaine, prilocaine, phenacaine, benzocaine, diperodon, and combinations thereof. Each possibility represents a separate embodiment of the present invention.

In some embodiments, said anesthetic agent is pramoxine.

In some embodiments, said vasoconstrictor is selected from the group consisting of phenylephrine, an amphetamine, an antihistamine, methylphenidate, mephedrone, oxymetazoline, pseudoephedrine, psilocybin, and combinations thereof. Each possibility represents a separate embodiment of the present invention.

In some embodiments, said vasoconstrictor is phenylephrine.

In some embodiments, said pharmaceutical agents comprise a combination of pramoxine and phenylephrine.

In some embodiments, said pharmaceutical agent is selected from the group consisting of an immunomodulator, a toxin, a muscle relaxant, an antipruritic agent, an anti-inflammatory agent, an antibiotic agent, an antioxidant, and combinations thereof. Each possibility represents a separate embodiment of the present invention.

In some embodiments, said immunomodulator is selected from the group consisting of an imidazoquinoline, an imidazopyridine, an imidazonaphthyridine, derivatives and combinations thereof. Each possibility represents a separate embodiment of the present invention.

In some embodiments, said immunomodulator is selected from the group consisting of imiquimod and resiquimod. Each possibility represents a separate embodiment of the present invention.

In some embodiments, said toxin is podophyllotoxin or podophyllin. Each possibility represents a separate embodiment of the present invention.

In some embodiments, said pharmaceutical liquid adhesive further comprises a dispersing agent.

In some embodiments, said dispersing agent is a non-volatile siloxane containing polymer.

In some embodiments, said non-volatile siloxane containing polymer is polyphenylmethylsiloxane.

In some embodiments, said dispersing agent is a silicone surfactant.

In some embodiments, said silicone surfactant is selected from the group consisting of polyalkyl modified silicone oil, polyether modified silicone oil, and polyalkyl and polyether modified silicone oil. Each possibility represents a separate embodiment of the present invention.

In some embodiments, said pharmaceutical liquid adhesive composition comprises trimethylsiloxysilicate; hexamethyldisiloxane; dispersing agent; and at least one pharmaceutical agent.

In some embodiments, said pharmaceutical liquid adhesive composition comprises trimethylsiloxysilicate; isooctane; dispersing agent; and at least one pharmaceutical agent.

In some embodiments, said pharmaceutical liquid adhesive composition comprises trimethylsiloxysilicate; hexamethyldisiloxane; isooctane; dispersing agent; and at least one pharmaceutical agent.

In some embodiments, said pharmaceutical liquid adhesive composition comprises about 10-40% w/w trimethylsiloxysilicate; about 55-70% w/w of hexamethyldisiloxane and isooctane; about 2-14% w/w polyphenylmethylsiloxane; and at least one pharmaceutical agent.

In some embodiments, said pharmaceutical liquid adhesive composition comprises about 10-40% w/w trimethylsiloxysilicate; about 55-70% w/w of hexamethyldisiloxane and isooctane; about 2-14% w/w polyalkyl and/or polyether modified silicone oil; and at least one pharmaceutical agent.

In some embodiments, said pharmaceutical liquid adhesive composition further comprises siloxane containing monomers.

In some embodiments, said siloxane containing monomers are hydrogen dimethicone with vinyldimethicone; bis-vinyldimethicone; and any combination thereof. Each possibility represents a separate embodiment of the present invention.

In some embodiments, said pharmaceutical liquid adhesive composition further comprises silicone gum blend.

In some embodiments, said silicone gum blend comprises cyclopentasiloxane and dimethiconol.

In some embodiments, said pharmaceutical liquid adhesive composition comprises about 15% w/w trimethylsiloxysilicate; about 25% w/w hexamethyldisiloxane and 32% isooctane; about 5% w/w polyphenylmethylsiloxane; about 4% w/w polyalkyl and polyether modified silicone oil; about 5% w/w vinyldimethicone and hydrogen dimethicone and 5% w/w bis-vinyldimethicone; about 1% w/w cyclopentasiloxane and dimethiconol; about 1% w/w pramoxine; and about 0.05% w/w phenylephrine. Each possibility represents a separate embodiment of the present invention.

In some embodiments, said pharmaceutical liquid adhesive composition further comprises a low hydrophile-lipophile balance surfactant.

In some embodiments, said low hydrophile-lipophile balance surfactant is micronized.

In some embodiments, said low hydrophile-lipophile balance surfactant is glyceryl monooleate (GMO).

In some embodiments, said low hydrophile-lipophile balance surfactant is sorbitan monooleate (SPAN 80).

In some embodiments, said pharmaceutical liquid adhesive composition comprises 4 to 10% by weight low hydrophile-lipophile balance surfactant.

The present invention further provides, in an aspect, a method of preventing or treating an anorectal disorder, the method comprising the step of topically applying to the mucosal surface of an anorectal region of a subject in need of such treatment a therapeutically effective amount of the liquid adhesive composition described above.

In some embodiments, said anorectal disorder is selected from the group consisting of hemorrhoids, anal fissures, anal cracks, anal fistulas, anal abscesses, anal warts, and anal pruritis. Each possibility represents a separate embodiment of the present invention.

In some embodiments, said anorectal disorder is hemorrhoid.

In some embodiments, said subject is a human subject or an animal.

The present invention further provides, in another aspect, a kit comprising a pharmaceutical liquid adhesive composition described above, and a container-applicator device suitable for storage and application of said composition to the rectum or anal canal.

In some embodiments, said container-applicator device comprises at least one of a single use wipe, a syringe, a dropper, a spray dispenser, a compressible bottle or tube, a spatula, a suppository insertion tube, an extrusion tube, and an inflatable member. Each possibility represents a separate embodiment of the present invention.

The present invention further provides, in another aspect, a pharmaceutical liquid adhesive composition described above, for use in preventing or treating an anorectal disorder. Each possibility represents a separate embodiment of the present invention.

The present invention further provides, in another aspect, a method for preparing a pharmaceutical liquid adhesive composition described above, comprising the steps of preparing a first mixture comprising a silicone film forming agent such as trimethylsiloxysilicate powder; a volatile solvent such as hexamethyldisiloxane and/or isooctane, a siloxane containing monomer such as bis-vinyldimethicone, vinyldimethicone and hydrogen dimethicone, and a gum blend comprising cyclopentasiloxane and dimethicone; preparing a second mixture by means of a homogenizer, said mixture comprising a pharmaceutical agent such as pramoxine and phenylephrine; and combining said first and second mixtures by means of a homogenizer.

In some embodiments, said second mixture further comprises a non-volatile siloxane such as polyphenylmethylsiloxane, and a silicone surfactant such as polyalkyl and polyether modified polydimethylsiloxane. Each possibility represents a separate embodiment of the present invention.

In some other embodiments, said second mixture further comprises a low hydrophile-lipophile balance surfactant such as glyceryl monooleate (GMO) and Sorbitan monooleate (SPAN-80). Each possibility represents a separate embodiment of the present invention.

Other objects, features and advantages of the present invention will become clear from the following description and claims.

### DETAILED DESCRIPTION OF THE INVENTION

### Liquid Adhesive

The present invention provides, in an aspect, a pharmaceutical liquid adhesive composition comprising a silicone film forming agent; a volatile solvent selected from the group consisting of a volatile polydimethylsiloxane, a volatile aliphatic hydrocarbon and a mixture thereof; and at least one pharmaceutical agent.

The term “pharmaceutical liquid adhesive composition” as used herein refers to a composition comprising at least one active ingredient.

The non-limiting examples of a film forming agent in accordance with the present invention are trimethylsiloxysilicate, polymethylsilsesquioxane, and mixtures thereof. The preferred film forming agent is trimethylsiloxysilicate. Trimethylsiloxysilicate is widely used in cosmetic industry due to its film forming properties, as described, for example, in U.S. Pat. Nos. 7,879,316 and 7,879,346. The present invention discloses for the first time the use of trimethylsiloxysilicate for therapeutic applications, inter alia, for treatment of anorectal disorders. Trimethylsiloxysilicate is soluble in the liquid components of the liquid adhesive composition. The amount of the silicone film forming agent in the composition is determined based on the desired adhesion properties of the film to the target surface. The amount depends, inter alia, on the target surface, the condition to be treated, and the amount of composition ingredients. The amount of the silicone film forming agent in the composition typically ranges from about 10% to 40% w/w.

The liquid adhesive compositions of the present invention comprise at least one solvent. The solvent dissolves the trimethylsiloxysilicate powder and enables homogeneous mixture of the liquid adhesive composition. Upon application of the liquid adhesive to the target surface, the solvent evaporates, leaving an adhered film which comprises at least one active agent. It is to be appreciated that the compositions of the present invention are devoid of polar solvents required for dissolving active ingredients, thus providing non-stinging liquid adhesives that have a comfortable feel when applying on the mucosal anal/genital surface.

The liquid adhesive composition can comprise a volatile polydimethylsiloxane. Certain non-limiting examples of polydimethysiloxanes in accordance with the present invention are hexadimethyl disiloxane (HDMS), octamethyl cyclotetrasiloxane, decamethyl cyclopentasiloxane, octamethyl trisiloxane, and mixtures thereof. The preferred polydimethylsiloxane used in the compositions is HMDS.

In some embodiments, said silicone film forming agent is trimethylsiloxysilicate.

The liquid adhesive can further comprise a volatile aliphatic hydrocarbon. The aliphatic hydrocarbon in accordance with the present invention may be any aliphatic hydrocarbon, including an alkane, a mixture of alkanes, an alkene, a mixture of alkenes, an alkyne, a mixture of alkynes, or a mixture thereof. The aliphatic hydrocarbon is preferably an alkane such as pentane or isooctane, or a mixture thereof. According to a certain embodiment, the aliphatic hydrocarbon is isooctane.

The liquid adhesive composition can comprises a volatile polydimethylsiloxane, a volatile aliphatic hydrocarbon or a mixture thereof. According to a certain embodiment, the volatile solvent comprises hexamethyl disiloxane and isooctane.

The amount of the volatile solvent affects the viscosity and solvent evaporation time of the liquid adhesive composition when applied to a target surface. The amount of the volatile solvent can be determined by a person skilled in art so as to adjust the viscosity and evaporation time to desired values. The amount of the volatile solvent in the composition is typically ranges from about 60%-70% w/w.

The term “about” as used herein denotes ±10% of the value indicated.

In some embodiments, said volatile polydimethylsiloxane is selected from the group consisting of hexamethyldisiloxane, octamethyl cyclotetrasiloxane, decamethyl cyclopentasiloxane, octamethyl trisiloxane, and mixtures thereof. Each possibility represents a separate embodiment of the present invention.

In some embodiments, said volatile aliphatic hydrocarbon is selected from the group consisting of alkanes, alkenes, alkynes, and mixtures thereof. Each possibility represents a separate embodiment of the present invention.

In some embodiments, said alkane is selected from the group consisting of pentane, isooctane, and mixtures thereof. Each possibility represents a separate embodiment of the present invention.

The liquid adhesive composition of the present invention comprises at least one pharmaceutically active agent, such as an anesthetic, an antibiotic, an immunomodulator, a muscle relaxant, a toxin, a vasoconstrictor, an antipruritic agent, or an antioxidant. The composition may further contain one or more additional pharmaceutical active agents, excipients and carriers. Additional pharmaceutical active agents include for example, analgesics, antimicrobial agents and botanical products or extracts. Pharmaceutically and dermatologically acceptable excipients and carriers as are known in the art may be included in the composition, in particular for maintaining the stability and sterility of the composition, and for promoting delivery, release and/or application of the active agent(s) to the body surface to which the composition is applied.

It is to be understood that the compositions may contain more than one active agent, and/or may be suitable for use in treating different anorectal or genital disorders. In a currently preferred embodiment, the composition comprises an anesthetic agent and a vasoconstrictor. Exemplary anesthetic agent is pramoxine. Pharmaceutically acceptable salts of the aforementioned anesthetic agents may also be included in the composition of the invention. Suitable amounts of such anesthetic agents in the composition may be readily ascertained by one of ordinary skill in the art, and may range, for example, between 0.25% and 25% by weight. In a particular embodiment, the anesthetic agent is pramoxine HCl or lidocaine. In a particular embodiment, the composition of the invention comprises pramoxine HCl at a concentration of 1% w/w based on the total weight of the composition.

Vasoconstrictors which are suitable for use in the invention include amphetamines, antihistamines, methylphenidate, mephedrone, oxymetazoline, phenylephrine, pseudoephedrine and psilocybin. Vasoconstrictor agents include, but are not limited to, phenylephrine hydrochloride, ephedrine sulphate, epinephrine, epinephrine hydrochloride and tetrahydrozoline HCl, and combinations thereof. Exemplary vasoconstrictor agent is pramoxine. In a particular embodiment, the composition of the invention comprises phenylephrine HCl at a concentration of about 0.05% w/w based on the total weight of the composition.

Immunomodulators, also known as immune response modifiers (IRMs), can also be used. Certain IRMs are known to be useful for treating viral diseases (e.g., human papilloma virus, herpes).

Immunomodulators which are suitable for use in the invention include small organic molecules such as imidazoquinoline amine derivatives, as disclosed for example in U.S. Pat. No. 4,689,338. IRMs of various other compound classes may also be used, as disclosed for example in U.S. Pat. Nos. 4,689,338; 5,482,936; 5,756,747; 6,110,929; 6,541,485; 6,756,382.

In particular embodiments, the immunomodulator may be selected from imidazoquinolines, imidazopyridines, imidazonaphthyridines, substituted derivatives thereof and combinations thereof.

In particular embodiments, the immunomodulator may be selected from imidazoquinoline amines, tetrahydroimidazoquinoline amines, imidazopyridine amines, 6,7-fused cycloalkylimidazopyridine amines, 1,2-bridged imidazoquinoline amines, imidazonaphthyridine amines, tetrahydroimidazonaphthyridine amines, oxazoloquinoline amines, thiazoloquinoline amines, oxazolopyridine amines, thiazolopyridine amines, oxazolonaphthyridine amines, thiazolonaphthyridine amines, 1H-imidazo dimers fused to pyridine amines, quinoline amines, tetrahydroquinoline amines, naphthyridine amines, or tetrahydronaphthyridine amines, and substituted derivatives thereof and combinations thereof.

In a particular embodiment, the immunomodulator for use in the invention is imiquimod, known chemically as 1-(2-methylpropyl)-1H-imidazo[4,5-c]quinolin-4-amine. In a particular embodiment, the immunomodulator for use in the invention is resquimod, known chemically as 1-[4-amino-2-(ethoxymethyl)-1H-imidazo[4,5-C]quinolin-1-yl]-2-methylpropan-2-ol.

Toxins which are suitable for use in the invention include a compound or mixtures of compounds derived from a plant, or synthetic equivalents thereof. In a particular embodiment, the toxin is selected from the group consisting of podophyllotoxin or podophyllin.

Muscle relaxants which are suitable for use in the invention include nitroglycerin, nifedipene, amlodopine, sildenafil, tizanidine, and baclofen, or salts thereof including, but not limited to, sildenafil citrate.

Antipruritic agents which are suitable for use in the invention include topical corticosteroids, camphor, juniper tar and menthol. The non-limiting examples of topical corticosteroids include hydrocortisone, fluocinolone, flurandrenolide, triamcinolone, fluticasone, and desonide.

Anti-inflammatory agents include salicylic acid, indomethacin, sodium indomethacin trihydrate, salicylamide, naproxen, colchicine, fenoprofen, sulindac, diflunisal, diclofenac, indoprofen and sodium salicylamide.

Antibiotics for use in the invention are preferably those suitable for topical application. The antibiotic(s) may be classified in one or more of the following groups: penicillins, cephalosporins, carbepenems, beta-lactam antibiotics, aminoglycosides, amphenicols, ansamycins, macrolides, lincosamides, glycopeptides, polypeptides, tetracylines, chloramphenicol, quinolones, fucidins, sulfonamides, sulfones, nitrofurans, diaminopyrimidines, trimethoprims, rifamycins, oxalines, streptogramins, lipopeptides, ketolides, polyenes, azoles, and echinocandins.

Specific examples of antibiotics which are suitable for use in the invention include: amikacin, aminosidine, paromomycin, chloramphenicol, ciprofloxacin, clindamycin, colistimethate-sodium, colistin, enfuvirtid, enoxacin, erythromycin, flucloxacillin, fosfomycin, fusafungin, gentamicin, levofloxacin, linezolid, mefloquin, metronidazol, mezlocillin, moxifloxacin, mupirocin, norfloxacin, ofloxacin, oxacillin, penicillin G, penicillin V, phenoxymethylpenicillin, phenoxymethylpenicillin-benzathin, pipemidinic acid, piperacillin, piperacillin+tazobactam, proguanil, propicillin, pyrimethamine, retapamulin, rifaximin, roxithromycin, sodium sulfacetamide, sulbactam, sulbactam+ampicillin, sulfadiazine, spiramycin, sultamicillin, tazobactam+piperacillin, teicoplanin, telithromycin, tigecyclin, vancomycin and combinations thereof.

Antioxidative compounds may also be included in the composition, in particular the antioxidative compounds collectively termed catechins. These include for example, epicatechin, epicatechin gallate, epigallocatechin gallate, and gallocatechin, as well as stereoisomers and enantiomers of these compounds and combinations thereof. Such compounds may be provided as synthetic compounds or in the forms of mixtures as components of plant extracts, in particular green tea extracts. Botanical products and extracts include those derived from peppermint, ginger horseradish, yarrow, chamomile, rosemary, capsicum, aloe vera, tea trea oil (melaleuca oil), among many others.

The present composition may include one or more of the following additional ingredients: emulsifiers (e.g. anionic, cationic or nonionic), chelating agents, colorants, emollients, fragrances, humectants, lubricants, moisturizers, preservatives, skin penetration enhancers, stabilizers, surfactants, thickeners, and viscosity modifiers.

In some embodiments, said pharmaceutical agent is selected from the group consisting of an anesthetic agent, a vasoconstrictor, and combinations thereof. Each possibility represents a separate embodiment of the present invention.

In some embodiments, said anesthetic agent is selected from the group consisting of pramoxine, procaine, lidocaine, tetracaine, dibucaine, prilocaine, phenacaine, benzocaine, diperodon, and combinations thereof. Each possibility represents a separate embodiment of the present invention.

In some embodiments, said anesthetic agent is pramoxine.

In some embodiments, said vasoconstrictor is selected from the group consisting of phenylephrine, an amphetamine, an antihistamine, methylphenidate, mephedrone, oxymetazoline, pseudoephedrine, psilocybin, and combinations thereof. Each possibility represents a separate embodiment of the present invention.

In some embodiments, said vasoconstrictor is phenylephrine.

In some embodiments, said pharmaceutical agent is pramoxine, phenylephrine or a combination of pramoxine and phenylephrine. Each possibility represents a separate embodiment of the present invention.

In some embodiments, said pharmaceutical agent is selected from the group consisting of an immunomodulator, a toxin, a muscle relaxant, an antipruritic agent, an anti-inflammatory agent, an antibiotic agent, an antioxidant, and combinations thereof. Each possibility represents a separate embodiment of the present invention.

In some embodiments, said immunomodulator is selected from the group consisting of an imidazoquinoline, an imidazopyridine, an imidazonaphthyridine, derivatives and combinations thereof. Each possibility represents a separate embodiment of the present invention.

In some embodiments, said immunomodulator is selected from the group consisting of imiquimod and resiquimod. Each possibility represents a separate embodiment of the present invention.

In some embodiments, said toxin is podophyllotoxin or podophyllin. Each possibility represents a separate embodiment of the present invention.

The liquid adhesive composition further comprises a silicone dispersing agent. As used herein, the term “dispersing agent” refers to an agent which causes the active pharmaceutical agent to be substantially homogeneously distributed in the composition according to some embodiments of the invention.

The silicone dispersing agent may be a non-volatile siloxane containing polymer. Without being bound to any mechanism of action, the addition of the non-volatile siloxane containing polymer to the composition can prevent pharmaceutical agents from floating in the volatile solvent and allows their homogeneous dispersion in the liquid adhesive solution. The existence of the siloxane containing polymer in the solution further allows the transfer of the solution to the intended container-applicator device, e.g. a wipe, retaining fine dispersion of the active ingredients. Upon application of the liquid adhesive to the target area and evaporation of the volatile solvent, the non-volatile siloxane containing polymer remains in the formed film, enhancing its silkiness. The non-volatile siloxane containing polymer can be a polydiorganosiloxane. The polyorganosiloxane is selected from the group consisting of: polydimethylsiloxane, polyphenylmethylsiloxane, poly(diphenylsiloxane dimethylsiloxane), poly(dimethylsiloxane methylvinylsiloxane), poly(dimethylsiloxane phenylmethylsiloxane), and poly(diphenylsiloxane dimethylsiloxane methylvinylsiloxane). The preferred non-volatile siloxane containing polymer of the liquid adhesive composition is polymethylphenylsiloxane. Polymethylphenylsiloxanes are available, for example, from Dow Corning as 556 Cosmetic Grade Fluid™ or from the General Electric Company as SP-1075 methyl phenyl fluid™. The amount of the non-volatile siloxane containing polymer in the composition is determined based on the pharmaceutical agents and their amounts and should be adjusted to obtain the desired dispersion. The non-volatile siloxane containing polymer is present in the composition in an amount ranging from about 1% to about 14% w/w.

The silicone dispersing agent in the liquid adhesive composition of the invention may alternatively be a silicone surfactant. Without being bound to any mechanism of action, the addition of the silicone surfactant can prevent pharmaceutical agents from clamping and allows their homogeneous dispersion in the liquid adhesive solution. The silicone surfactant is selected from the group consisting of polyalkyl modified silicone oil, polyether modified silicone oil, and polyalkyl and polyether modified silicone oil. The amount of the silicone surfactant in the composition is determined based on the pharmaceutical agents and their amounts and should be adjusted to obtain the desired dispersion. The silicone surfactant is present in the composition in an amount ranging from about 1% to 4% w/w.

The silicone dispersing agent may further comprise a combination of the non-volatile siloxane containing polymer and the silicone surfactant. The liquid adhesive of the present invention can comprise about 5% w/w polyphenylmethylsiloxane and 4% w/w polyalkyl and polyether modified silicone oil.

In some embodiments, said pharmaceutical liquid adhesive further comprises a dispersing agent.

In some embodiments, said dispersing agent is a non-volatile siloxane containing polymer.

In some embodiments, said non-volatile siloxane containing polymer is polyphenylmethylsiloxane.

In some embodiments, said dispersing agent is a silicone surfactant.

In some embodiments, said silicone surfactant is selected from the group consisting of polyalkyl modified silicone oil, polyether modified silicone oil, and polyalkyl and polyether modified silicone oil. Each possibility represents a separate embodiment of the present invention.

In some embodiments, said pharmaceutical liquid adhesive composition comprises trimethylsiloxysilicate; hexamethyldisiloxane; dispersing agent; and at least one pharmaceutical agent.

In some embodiments, said pharmaceutical liquid adhesive composition comprises trimethylsiloxysilicate; isooctane; dispersing agent; and at least one pharmaceutical agent.

In some embodiments, said pharmaceutical liquid adhesive composition comprises trimethylsiloxysilicate; hexamethyldisiloxane; isooctane; dispersing agent; and at least one pharmaceutical agent.

In some embodiments, said pharmaceutical liquid adhesive composition comprises about 10-40% w/w trimethylsiloxysilicate; about 55-70% w/w of hexamethyldisiloxane and isooctane; about 2-14% w/w polyphenylmethylsiloxane; and at least one pharmaceutical agent.

In some embodiments, said pharmaceutical liquid adhesive composition comprises about 10-40% w/w trimethylsiloxysilicate; about 55-70% w/w of hexamethyldisiloxane and isooctane; about 2-14% w/w polyalkyl and/or polyether modified silicone oil; and at least one pharmaceutical agent.

The liquid adhesive may further comprise siloxane containing monomers. Without being bound to any mechanism of action, the addition of siloxane containing monomers to the liquid adhesive composition can provide enhanced film adhesion onto the target surface and can allow reduction of skin strain, which may be caused by trimethylsiloxysilicate. The monomers form a cross-polymer upon evaporation of the volatile solvents of the liquid adhesive, enhancing the composition adhesive properties. The siloxane containing monomers can be selected from dimethicone, hydrogen dimethicone, vinyldimethicone and bis-vinyldimethicon. Each possibility is a separate embodiment of the invention. The preferred liquid adhesive composition comprises (hydrogen dimethicone and vinyldimethicone) and bis-vinyldimethicon.

In some embodiments, said pharmaceutical liquid adhesive composition further comprises siloxane containing monomers.

In some embodiments, said siloxane containing monomers are hydrogen dimethicone with vinyldimethicone; bis-vinyldimethicone; and any combination thereof. Each possibility represents a separate embodiment of the present invention.

The liquid adhesive may further comprise a silicone gum blend. Without being bound to any mechanism of action, the addition of the silicone gum blend provides enhancement of silkiness of the film. One non-limiting example of a gum blend in accordance with the present invention is cyclopentasiloxane and dimethiconol. The cyclopentasiloxane and dimethiconol blends are available, for example, from KCC as SF9902E™ or from Momentive as Silsoft 1215 dimethicone™.

In some embodiments, said pharmaceutical liquid adhesive composition further comprises silicone gum blend.

In some embodiments, said silicone gum blend comprises cyclopentasiloxane, dimethiconol or cyclopentasiloxane and dimethiconol. Each possibility represents a separate embodiment of the present invention.

In some embodiments, said pharmaceutical liquid adhesive composition comprises about 15% w/w trimethylsiloxysilicate; about 25% w/w hexamethyldisiloxane and 32% isooctane; about 5% w/w polyphenylmethylsiloxane; about 4% w/w polyalkyl and polyether modified silicone oil; about 5% w/w vinyldimethicone and hydrogen dimethicone and 5% w/w bis-vinyldimethicone; about 1% w/w cyclopentasiloxane and dimethiconol; about 1% w/w pramoxine; and about 0.05% w/w phenylephrine. Each possibility represents a separate embodiment of the present invention.

In some embodiments, said pharmaceutical liquid adhesive composition further comprises a low hydrophile-lipophile balance surfactant.

In some embodiments, said low hydrophile-lipophile balance surfactant is micronized.

In some embodiments, said low hydrophile-lipophile balance surfactant is glyceryl monooleate (GMO).

In some embodiments, said low hydrophile-lipophile balance surfactant is sorbitan monooleate (SPAN 80).

In some embodiments, said pharmaceutical liquid adhesive composition comprises 4 to 10% by weight low hydrophile-lipophile balance surfactant. In some embodiments, said pharmaceutical liquid adhesive composition comprises 4, 5, 6, 7, 8, 9 or 10% by weight low hydrophile-lipophile balance surfactant. Each possibility represents a separate embodiment of the present invention.

The present invention further provides, in an aspect, a method of preventing or treating an anorectal disorder, the method comprising the step of topically applying to the mucosal surface of an anorectal region of a subject in need of such treatment a therapeutically effective amount of the liquid adhesive composition described above.

In some embodiments, said anorectal disorder is selected from the group consisting of hemorrhoids, anal fissures, anal cracks, anal fistulas, anal abscesses, anal warts, anal pruritis. Each possibility represents a separate embodiment of the present invention.

In some embodiments, said anorectal disorder is hemorrhoid.

In some embodiments, said subject is a human subject or an animal.

### Containers and Applicators

The compositions for use in the present invention are generally stored in a container-applicator device for use in a single dose application (e.g., a wipe or a swab in a disposable container) or for use in repeated applications to the anal canal. Single dose applicators include those having breakable or removable seals that prevent moisture, including atmospheric moisture, from contacting the formulation.

In the preferred embodiment of the invention, the liquid adhesive is comprised in the pre-packaged towelette/wipe. The wipe substrate is typically uniformly impregnated with the liquid adhesive composition. The wipe provides the user with a single dose of sterile medication. The liquid adhesive is transferred to the body surface upon contacting the wipe with the target surface.

A container-applicator may comprise two parts: (1) a storage area or reservoir which holds the composition and protects it from air, water and contaminants; and (2) the applicator which generally comprises a specially shaped tip designed to aid in application of the composition to the anal and/or rectal mucosa. In particular embodiments, the applicator is an element integral to the container, for example, an elongated insertion tube extending from a reservoir. Alternately, the storage area and the applicator may be separate components, such as a tube reservoir and a separately supplied dropper. In yet other embodiments, the container and the applicator may be supplied as separate elements which are connected during use, for example via compatible male and female connectors respectively provided on the container and the applicator or vice versa.

For repeated and intermittent usage, minimal exposure to atmospheric moisture is required. This can be achieved by devices having very narrow applicator outlets and low initial dead space. One applicator for such repeated intermittent use preferably dispenses the adhesive in a controlled drop wise manner, as described for example in U.S. Pat. No. 4,958,748.

Still another container-applicator device comprises a brush or solid paddle applicator wherein the liquid adhesive is “painted” onto the surface requiring treatment.

An exemplary container-applicator device for repeated and intermittent usage comprises a container suitable for non-sterile storage of the composition, and an applicator suitable for metered dispersement of the composition after opening of the applicator. In particular embodiments, the applicator is characterized as having a resealable opening of no more than about 0.05 square inch (0.323 square centimeters) so as to permit the metered dispersement of the adhesive from the applicator and which is capable of multiple administrations of the adhesive, and is further characterized as having resealing means such as a cap which either tightly mates with the applicator or which screws onto the applicator. The opening may be at the terminus of an elongated and tapered tube-like member suitable for insertion into the anal canal and accessing internal hemorrhoids. Preferably, the opening of the applicator is about 0.001 to about 0.01 square inch (about 0.00645 to about 0.0645 square centimeters).

In another embodiment, the walls of the container-applicator device are made of a pliable material, so that upon application of pressure onto the walls, the walls depress sufficiently to force the adhesive in the container into the applicator and through the opening. In another embodiment, the adhesive is released from the applicator by gravity feed methods well known in the art. Such methods do not require application of pressure to the walls of the container.

Preferably, the applicator is manufactured with its opening covered by a metal foil or other similar construction which closes this opening until the device is ready for use. The opening is then reinstated by use of a pin or similar device which punctures the covering.

Such devices for intermittent use enable multiple uses of the liquid adhesive at different points in time by the same individual.

In container-applicator devices suitable for repeated intermittent uses, the liquid adhesive is stored at ambient conditions and is selected to be bacteriostatic. See, for example, U.S. Pat. No. 3,527,224. When the selected adhesive is bacteriostatic, prolonged storage at ambient conditions can be achieved without regard to the sterility of the formulation because there is no adverse buildup of bacteria during storage.

The reservoir of the container-applicator device is preferably both air-tight and water-tight, and keeps the media within free from contaminants. The reservoir may contain a desiccant material to keep the media free of water. Reservoirs may be of any shape, although shapes which provide for a smooth internal flow of media, such as cylindrical or conical shapes, are preferred. The size of the reservoir may vary within a wide range, but is preferably slightly larger than the volume of composition which will be placed inside the reservoir to minimize the amount of gas within the reservoir. The reservoir may be made from any of a variety of medical grade materials, such as plastics, excluding glass. Pharmaceutical agents of the liquid adhesive suffer from caking when stored in glass reservoir. The reservoir may be either rigid, collapsible, or compressible. Use of a compressible or collapsible reservoir allows the user to have greater control over the rate at which the composition is expressed, as exertion of pressure on a compressible or collapsible reservoir would place a force on the on the composition causing it to flow at a faster rate than it would in the absence of such pressure. The compressible or collapsible reservoir design is especially preferred for highly viscous or gel-like compositions for which the force of gravity may not be strong enough to cause a flow through an applicator sufficient to treat hemorrhoids or fissures. Collapsible reservoirs which retain their collapsed shape have the additional advantage of reducing the amount of air which enters the reservoir following use. This advantage of collapsible containers is of greater importance in multiple-use (reusable) devices, wherein media is preferably kept relatively free of potential contaminants between uses.

Applicator tips can be of any of a number of shapes, sizes, and configurations. They are preferably fairly rigid and may be made out of any material which is compatible with the media formulation, preferably plastic, excluding glass. The choice of a proper applicator tip for a given application will depend on factors such as the viscosity of the composition, the desired application rate of the composition, the nature of the anal disorder, and its severity.

The container-applicators of the present invention may be either single-use or multiple-use devices. A container or reservoir containing enough liquid adhesive composition for multiple applications may be configured to accommodate replaceable tips. In such an embodiment, at the place whereon the replaceable tips connect with the reservoir, the reservoir would preferably have a means such as a valve, septum or sealing gasket which allows the reservoir to be sealed in the absence of an applicator tip. Placing an applicator tip on the reservoir would cause the valve to open, allowing composition to flow out from the reservoir. In this manner, one reservoir containing enough composition for several applications could be used over a period of hours, days or weeks. This embodiment would also allow the user to use one reservoir with applicator tips of varying shapes and sizes chosen to best accommodate the anal disorder during the healing process.

The present invention thus further provides, in an aspect, a kit comprising a pharmaceutical liquid adhesive composition described above, and a container-applicator device suitable for storage and application of said composition to the rectum or anal canal.

In some embodiments, said container-applicator device comprises at least one of a single use wipe, a syringe, a dropper, a spray dispenser, a compressible bottle or tube, a spatula, a suppository insertion tube, an extrusion tube, and an inflatable member. Each possibility represents a separate embodiment of the present invention.

### Uses

Disorders of the anorectal region are commonly encountered among the general population, but are often inadequately unaddressed, since many patients delay or fail to seek medical attention due to embarrassment. Furthermore, many medications for such conditions fail to provide adequate relief and healing. In addition, many medications which are intended for treatment of conditions such as hemorrhoids and anal warts may be difficult to self-administer, and are unsatisfactory due to their uncomfortable sensation after application.

The present invention provides compositions which are useful for effectively treating a variety of anorectal disorders including hemorrhoids, anal fissures, anal cracks, anal fistulas, anal abscesses, anal warts, and anal pruritis, wherein the compositions provide enhanced therapeutic efficacy and are associated with improved patient compliance, as compared to prior art compositions. The provided compositions may be useful for simultaneously treating a number of anorectic disorders.

Hemorrhoids (also known as piles) form part of the normal human anatomy of the anal canal, but may become pathological when swollen or inflamed. In their physiological state they act as cushions composed of arterio-venous channels and connective tissue that aid the passage of stool. The symptoms of pathological hemorrhoids include rectal bleeding, tenderness and pain in the anal area.

Pathological hemorrhoids are typically classified as external or internal, which are differentiated via their position with respect to the dentate line. External hemorrhoids occur outside the anal verge (the distal end of the anal canal) as varicosities of the veins draining the territory of the inferior rectal arteries, which are branches of the internal pudendal artery. External hemorrhoids are frequently painful, and are often accompanied by swelling, skin irritation and itching. External hemorrhoids are prone to thrombosis, which may occur if the vein ruptures and/or a blood clot develops.

Internal hemorrhoids occur within the rectum as varicosities of veins draining the territory of branches of the superior rectal arteries. As this area lacks pain receptors, internal hemorrhoids are often painless and affected individuals may be unaware of their occurrence. Internal hemorrhoids may however, bleed when irritated. Untreated internal hemorrhoids can lead to the more sever conditions of prolapsed or strangulated hemorrhoids. Prolapsed hemorrhoids are severely distended such that they are extruded outside the anus. If the anal sphincter muscle goes into spasm and traps a prolapsed hemorrhoid outside the anal opening, the supply of blood is cut off, and the hemorrhoid becomes a strangulated hemorrhoid.

Internal hemorrhoids can be further graded by the degree of prolapse, in which Grade I is characterized by the absence of prolapse; Grade II is characterized by prolapse upon defecation but which reduce spontaneously; Grade III is characterized by prolapse upon defecation, which may be manually reduced; and Grade IV is characterized by prolapse which cannot be manually reduced.

An anal fissure is a crack or tear in the skin of the anal canal. Acute cases may be associated with severe periodic pain after defecation, while chronic cases are associated with less intense pain. Anal fissures usually extend from the anal opening and are usually located posteriorly in the midline. Fissure depth may be superficial or extend down to the underlying sphincter muscle. Most anal fissures are due to stretching of the anal mucosa beyond their capability. A common cause of non-healing chronic fissures is spasm of the internal anal sphincter muscle, resulting in impaired blood supply to the anal mucosa. The result is a non-healing ulcer, which may become infected by fecal bacteria.

Non-surgical conventional treatments for acute and chronic anal fissures are generally those used for hemorrhoids. Topically applied medications used for relaxation of the sphincter muscle include nitroglycerine, nifedipine, diltiazem, sildenafil citrate, and/or lidocaine. Surgical treatment procedures such as anal stretch (Lord's operation) or lateral sphincterotomy are aimed to decrease sphincter spasm. Another approach involves injection of botulinum toxin into the anal sphincter.

Anorectal or perianal abscess (also known as anal/rectal abscess, perianal/perirectal abscess) is an abscess occurring adjacent to the anus, due to infection at one of the anal crypts of Morgagni. Most cases are sporadic, although individuals with diabetes mellitus or Crohn's disease, or those undergoing chronic steroid treatment have increased risk and incidence. The condition is generally treated by surgery to drain the infection, followed by oral administration of antibiotics and possibly topical treatments. Anal abscess often leads to an anal fistula, which is the development of an infected channel within a gland between the anal canal and external skin near the anus or rectum. This condition also requires surgical treatment generally followed by administration of antibiotics.

Anal warts (also known as Condylomata acuminata, venereal warts, genital warts and anogenital warts) represents a highly contagious sexually transmitted disease caused by human papillomavirus (HPV). This disease has a high incidence, with one million new cases diagnosed in the U.S. each year. Topical treatments for anal/genital warts include anti-mitotic agents such as podphyllotoxin (also known as podofilox), chemical immunomodulating agents, such as imiquimod; and green tea extracts comprising sinecatechins and other components.

Anal pruritis (also known as pruritus ani or anusitis) is an irritation of the skin at the anus, associated with intensive urge to scratch the affected area. The condition may be idiopathic, or associated with various factors or co-existing conditions, including occult or overt fecal soiling, ingestion of certain foods, bacterial or fungal infection, hemorrhoids or additional co-existing anorectal disorders, and dermatological conditions, in particular allergic contact dermatitis or psoriasis. Treatment measures include enhanced hygiene, antibiotics or antifungal medications when infections are present, various creams and ointments, generally containing local anesthetics, vasoconstrictors, protectants or combinations thereof, and topical steroids. The composition is applied to areas of the anal canal or rectum affected by hemorrhoids, fissures, fistulae, cracks, warts or pruritis, under conditions suitable for film formation of the adhesive so as to form a protective coating and typically under non-sterile conditions. In general, sufficient amounts of liquid adhesive are employed to cover the entire affected mucosal surface area. The coating is preferably extended by at least about 1 centimeter and preferably by at least about 5 centimeters beyond the affected surface area.

The term “therapeutically effective amount” is that amount of the pharmaceutical agent which is sufficient to provide a beneficial effect to the subject to which the pharmaceutical agent is administered. More specifically, a therapeutically effective amount means an amount of the pharmaceutical agent effective to alleviate or ameliorate the symptoms of an anorectal/genital disorder of the subject being treated.

After an initial layer of liquid adhesive has been applied and the solvent has evaporated, providing an initial adhesive coating, a second layer may be applied over the initial film. Additional amounts of liquid adhesive can be applied as needed.

Sufficient liquid adhesive is preferably employed to form a coating of less than about 0.5 mm thick and more preferably at least about 0.1 mm thick. Such coatings can be formed by applying, for example, about 0.02 ml of liquid adhesive per square centimeter of affected surface area.

In general, the particular length of time required for film formation will vary depending on factors such as the amount of adhesive applied, the temperature of the rectal or anal mucosal area, the moisture content of the rectal or anal, the surface area for adhesive application, and the like. However, in a preferred embodiment, film formation is generally complete within about 10 to about 60 seconds. During this period, the person to whom application of the liquid adhesive has been made preferably minimizes actions and body movements thus allowing the adhesive to form a coating.

The liquid adhesive compositions preferably act at room temperature (20° C.). The films are conformable and comfortable and may be elastic and flexible. The films do not irritate the skin and mucous membrane during the application and in use after drying. The liquid adhesives are preferably substantially painless and easily removable substantially without pain. The dried films formed from the liquid adhesive compositions are also preferably substantially non-water sensitive and waterproof. The dried films formed from the liquid adhesive compositions comprise finely-dispersed pharmaceutical ingredients, which can be gradually released to the adhesion area.

The compositions of the present invention are applicable to both human patients and to non-human mammalian subjects such as in veterinary use, for example for treatment of canine, feline, equine, bovine, porcine and primate species.

The present invention further provides, in an aspect, a pharmaceutical liquid adhesive composition described above, for use in preventing or treating an anorectal disorder. Each possibility represents a separate embodiment of the present invention.

### Methods

The present invention further provides, in an aspect, a method for preparing a pharmaceutical liquid adhesive composition described above, comprising the steps of preparing a first mixture comprising a silicone film forming agent such as trimethylsiloxysilicate powder; a volatile solvent such as hexamethyldisiloxane and/or isooctane, a siloxane containing monomer such as bis-vinyldimethicone, vinyldimethicone and hydrogen dimethicone, and a gum blend comprising cyclopentasiloxane and dimethicone; preparing a second mixture by means of a homogenizer, said mixture comprising a pharmaceutical agent such as pramoxine and phenylephrine; and combining said first and second mixtures by means of a homogenizer.

In some embodiments, said second mixture further comprises a non-volatile siloxane such as polyphenylmethylsiloxane, and a silicone surfactant such as polyalkyl and polyether modified polydimethylsiloxane. Each possibility represents a separate embodiment of the present invention.

In some other embodiments, said second mixture further comprises a low hydrophile-lipophile balance surfactant such as glyceryl monooleate and span-80. Each possibility represents a separate embodiment of the present invention.

The following examples illustrate certain embodiments of the invention but are not meant to limit the scope of the claims in any way.

### EXAMPLE 1

The following formulations are prepared for use in treating hemorrhoids.

| Ingredient | g per 100 g product | g per 100 g product |
|---|---|---|
| Trimethylsiloxysilicate | 15 | 20 |
| Hexamethyldisiloxane | 25 | 62 |
| Isooctane | 32 | |
| Polyphenylmethylsiloxane | 5 | 5 |
| Polyalkyl and polyether | 4 | 5 |
| modified polydimethylsiloxane | | |
| Pramoxine | 1 | 1 |
| Phenylephrine | 0.05 | 0.05 |
| Bis-vinyldimethicone | 5 | |
| Vinyldimethicone and | 5 | |
| hydrogen dimethicone | | |
| Cyclopentasiloxane and | 1 | |
| dimethicone blend | | |

### EXAMPLE 2

Trimethylsiloxysilicate powder is mixed with hexamethyldisiloxane, isooctane, bis-vinyldimethicone, (vinyldimethicone and hydrogen dimethicone) and cyclopentasiloxane and dimethicone blend. Polyphenlmethylsiloxane and polyalkyl and polyether modified polydimethylsiloxane are mixed with pramoxine and phenylephrine by means of a homogenizer. The trimethylsilicate solution is combined with the pharmaceutical agents' solution and mixed by means of a homogenizer. The obtained liquid adhesive solution is applied to the wipe substrate and sealed to provide the sealed package of single-use wipe impregnated with the liquid adhesive. The formulation is applied using single use wipe, wiping the anal region of an adult subject suffering from external hemorrhoids.

### EXAMPLE 3

The following formulations are prepared for use in treating hemorrhoids.

| Ingredient | g per 100 g product |
|---|---|
| Trimethylsiloxysilicate | 15-20 |
| Hexamethyldisiloxane | 25-62 |
| Isooctane | 0-32 |
| Polyphenylmethylsiloxane | 0 |
| Polyalkyl and polyether | 0 |
| modified polydimethylsiloxane | |
| Glyceryl monooleate | 0-10^{a} |
| Sorbitan monooleate | 0-10^{a} |
| (SPAN-80) | |
| Pramoxine | 1 |
| Phenylephrine | 0.05 |
| Bis-vinyldimethicone | 0-5 |
| Vinyldimethicone and | 0-5 |
| hydrogen dimethicone | |
| Cyclopentasiloxane and | 0-1 |
| dimethicone blend | |

^{a}Glyceryl monooleate and/or sorbitan monooleate (SPAN-80) comprise 4 to 10% by weight of the final product.

### EXAMPLE 4

Trimethylsiloxysilicate powder is mixed with hexamethyldisiloxane, isooctane, bis-vinyldimethicone, (vinyldimethicone and hydrogen dimethicone) and cyclopentasiloxane and dimethicone blend. Glyceryl monooleate (GMO) and/or span-80 are mixed with pramoxine and phenylephrine by means of a homogenizer. The trimethylsilicate solution is combined with the pharmaceutical agents' solution and mixed by means of a homogenizer. The obtained liquid adhesive solution is applied to the wipe substrate and sealed to provide the sealed package of single-use wipe impregnated with the liquid adhesive. The formulation is applied using single use wipe, wiping the anal region of an adult subject suffering from external hemorrhoids.

The foregoing description of the specific embodiments will so fully reveal the general nature of the invention that others can, by applying current knowledge, readily modify and/or adapt for various applications such specific embodiments without undue experimentation and without departing from the generic concept, and, therefore, such adaptations and modifications should and are intended to be comprehended within the meaning and range of equivalents of the disclosed embodiments. It is to be understood that the phraseology or terminology employed herein is for the purpose of description and not of limitation. The means, materials, and steps for carrying out various disclosed functions may take a variety of alternative forms without departing from the invention.

1. A pharmaceutical liquid adhesive composition comprising:
(i) a silicone film forming agent comprising trimethylsiloxysilicate;
(ii) a volatile solvent selected from the group consisting of a volatile polydimethylsiloxane, a volatile aliphatic hydrocarbon and a mixture thereof; and
(iii) at least one pharmaceutical agent.

2. The pharmaceutical liquid adhesive composition according to claim 1, wherein said silicone film forming agent is trimethylsiloxysilicate.

3. The pharmaceutical liquid adhesive composition according to claim 1, wherein said volatile polydimethylsiloxane is selected from the group consisting of hexamethyldisiloxane, octamethyl cyclotetrasiloxane, decamethyl cyclopentasiloxane, octamethyl trisiloxane, and mixtures thereof.

4. The pharmaceutical liquid adhesive composition according to claim 1, wherein said volatile aliphatic hydrocarbon is selected from the group consisting of alkanes, alkenes, alkynes, and mixtures thereof.

5. The pharmaceutical liquid adhesive composition according to claim 4, wherein said alkane is selected from the group consisting of pentane, isooctane, and mixtures thereof.

6. The pharmaceutical liquid adhesive composition according to claim 1, wherein said pharmaceutical agent is selected from the group consisting of an anesthetic agent, a vasoconstrictor, and combinations thereof.

7. The pharmaceutical liquid adhesive composition according to claim 6, wherein said anesthetic agent is selected from the group consisting of pramoxine, procaine, lidocaine, tetracaine, dibucaine, prilocaine, phenacaine, benzocaine, diperodon, and combinations thereof.

8. The pharmaceutical liquid adhesive composition according to claim 7, wherein said anesthetic agent is pramoxine.

9. The pharmaceutical liquid adhesive composition according to claim 6, wherein said vasoconstrictor is selected from the group consisting of phenylephrine, an amphetamine, an antihistamine, methylphenidate, mephedrone, oxymetazoline, pseudoephedrine, psilocybin, and combinations thereof.

10. The pharmaceutical liquid adhesive composition according to claim 9, wherein said vasoconstrictor is phenylephrine.

11. The pharmaceutical liquid adhesive composition according to claim 1, wherein said pharmaceutical agent is a combination of pramoxine and phenylephrine.

12.-20. (canceled)

21. The pharmaceutical liquid adhesive composition according to claim 1, comprising:
(i) trimethylsiloxysilicate;
(ii) hexamethyldisiloxane;
(iii) dispersing agent; and
(iv) at least one pharmaceutical agent.

22. The pharmaceutical liquid adhesive composition according to claim 1, comprising:
(i) trimethylsiloxysilicate;
(ii) isooctane;
(iii) dispersing agent; and
(iv) at least one pharmaceutical agent.

23. The pharmaceutical liquid adhesive composition according to claim 1, comprising:
(i) trimethylsiloxysilicate;
(ii) hexamethyldisiloxane;
(iii) isooctane;
(iv) dispersing agent; and
(v) at least one pharmaceutical agent.

24. The pharmaceutical liquid adhesive composition according to claim 1, comprising:
(i) about 10-40% w/w trimethylsiloxysilicate;
(ii) about 55-70% w/w of hexamethyldisiloxane and isooctane;
(iii) about 2-14% w/w polyphenylmethylsiloxane; and
(iv) at least one pharmaceutical agent.

25. The pharmaceutical liquid composition adhesive composition according to claim 1, comprising:
(i) about 10-40% w/w trimethylsiloxysilicate;
(ii) about 55-70% w/w of hexamethyldisiloxane and isooctane;
(iii) about 2-14% w/w polyalkyl and/or polyether modified silicone oil; and
(iv) at least one pharmaceutical agent.

26.-29. (canceled)

30. The pharmaceutical liquid adhesive composition according to claim 1 comprising:
(i) about 15% w/w trimethylsiloxysilicate;
(ii) about 25% w/w hexamethyldisiloxane and 32% isooctane;
(iii) about 5% w/w polyphenylmethylsiloxane;
(iv) about 4% w/w polyalkyl and polyether modified silicone oil;
(v) about 5% w/w vinyldimethicone and hydrogen dimethicone and 5% w/w bis-vinyldimethicone;
(vi) about 1% w/w cyclopentasiloxane and dimethiconol;
(vii) about 1% w/w pramoxine; and
(viii) about 0.05% w/w phenylephrine.

31.-35. (canceled)

36. A method of preventing or treating an anorectal disorder, the method comprising the step of topically applying to the mucosal surface of an anorectal region of a subject in need of such treatment a therapeutically effective amount of the liquid adhesive composition according to claim 1.

37. The method according to claim 36, wherein said anorectal disorder is selected from the group consisting of hemorrhoids, anal fissures, anal cracks, anal fistulas, anal abscesses, anal warts, anal pruritis.

38. The method according to claim 37, wherein said anorectal disorder is hemorrhoids.

39.-45. (canceled)
